(19) United States
(12) Patent Application Publication
(10) Pub. No.: US 2022/0015742 A1
(43) Pub. Date: Jan. 20, 2022

(54) SYSTEMS AND METHODS FOR AUTOMATED IMAGE RECOGNITION OF IMPLANTS AND COMPOSITIONS WITH LONG-LASTING ECHOGENICITY

(71) Applicant: Contraline, Inc., Charlottesville, VA (US)

(72) Inventors: Gregory Grover, Charlottesville, VA (US); Kevin Eisenfrats, Charlottesville, VA (US); Suchi Patel, Charlottesville, VA (US)

(21) Appl. No.: 17/358,501

(22) Filed: Jun. 25, 2021

Related U.S. Application Data
(62) Division of application No. 16/104,701, filed on Aug. 17, 2018.
(60) Provisional application No. 62/546,718, filed on Aug. 17, 2017.

(51) Int. Cl. A61B 8/08; A61F 6/22; A61F 2/02; A61L 31/02; A61L 31/14; A61L 31/04; A61L 31/06; A61B 8/00; G06T 7/194; G06T 7/00; A61L 31/18; G06T 7/136; A61L 31/12; G06T 7/155; G06T 7/11; A61L 27/44

(52) U.S. Cl. CPC A61B 8/5223; G06T 2207/10132; A61B 8/085; A61B 8/481; A61F 6/22; A61F 2/02; A61L 31/024; A61L 31/145; A61L 31/146; A61L 31/048; A61L 31/06; A61L 31/042; A61L 31/044; A61L 31/045; A61L 31/046; A61L 31/047; A61L 31/148; A61B 8/469; G06T 7/194; G06T 7/0012; A61L 31/18; G06T 7/136; A61L 31/126; G06T 7/155; G06T 7/11; A61L 27/443; A61F 2250/0096; G06T 2207/20036; G06T 2207/30052; A61L 2400/12; G06T 2207/20081; A61B 8/0841

(57) ABSTRACT

Systems and methods for imaging an object that are capable of capturing an image or images of the object using an imaging modality, automatically detecting and analyzing the image or images by way of converting the image or images to at least one binary image, and analyzing the at least one binary image to extract and/or segment regions-of-interest (ROIs) from the at least one binary image. The object can be or include an implantation, occlusion, medical device, body lumen, tissue, organ, duct, and/or vessel. The imaging modality can be or include X-ray, CT, MRI, PET, and/or ultrasound, or any combination thereof. Also included are compositions of soft, implantable materials with one or more carbon-based material, nanomaterial, and/or allotrope present in an amount sufficient as an ultrasound contrast agent effective for days, months, or years and which compositions are useful in the automated imaging methods of the invention.

### CROSS-REFERENCE TO RELATED APPLICATIONS

The present application is a Divisional Application of U.S. patent application Ser. No. 16/104,701, filed Aug. 17, 2018, which application published as U.S. Patent Publication No. 2019/0053790 on Feb. 21, 2019, which application relies on the disclosure of and claims priority to and the benefit of the filing date of U.S. Provisional Application No. 62/546,718, filed Aug. 17, 2017, the disclosures of which are hereby incorporated by reference herein in their entireties.

### BACKGROUND OF THE INVENTION

### Field of the Invention

The invention relates to the field of image processing and imageable compositions. Specifically, embodiments are directed to automated identification and analyzation of bodily implants by way of one or more medical imaging modality. Embodiments are also directed to determining information regarding an implant's brightness, echogenicity, location, density, volume, and/or homogeneity, along with other measurements and properties, as well as directed to compositions and methods for implanting soft materials that are echogenic, or ultrasound visible, and where the echogenicity of the material lasts as long as the implant itself.

### Description of Related Art

The field of “computer vision” is continually advancing in terms of scope and technology. Within this field, medical image analysis has emerged as an application with massive potential and constructive application, yet such analysis poses significant problems and limitations. Consequently, new methods of extracting and analyzing medical images within regions of interest (ROIs) are needed.

For example, ultrasound imaging allows for visualization of soft tissues based on differences in their echogenicity. Echogenicity of tissue is the ability of tissue to transmit or reflect ultrasound waves (USW) in relation to the surrounding tissues. When different tissues or tissue interfaces have differences in echogenicity, a contrast is created. A hyperechoic structure appears as white, a hypoechoic appears gray, and an anechoic appears black. Bone, for example appears black with a bright white rim because the USW cannot penetrate the bone. Cartilage appears hypoechoic since it is more transparent to USW than bone. Directional flow of fluids within vessels can be visualized based on color using modes such as Doppler. Structures within tissues are also visible. Muscles have striated features and are hypoechoic, while fat is unstructured and anechoic. Connective tissues, such as fascia and fascicles are hyperechoic and appear as lines. As an imaging diagnostic tool, contrast is an important feature in the context of ultrasound for imaging healthy versus unhealthy tissue.

It is common in the field to inject materials known as ultrasound contrast agents (UCAs) into tissue or intravenously to aid in visualizing specific tissue, areas, or compositions by enhancing the contrast or echogenic difference. UCAs can be traced back to the work of Garmiak and Shah in 1968. During an angiography, when injecting indocyanine they observed increased contrast, and this observation was traced back to air bubbles created at the needle tip by cavitation. This has since lead to work to stabilize these bubbles, herein known as microbubbles, and utilize them for a variety of ultrasound imaging purposes. Microbubbles are essentially bubbles of various gases with a shell made of materials such as fatty acids, proteins, mono or polysaccharides, and polymers. Microbubbles oscillate when exposed to ultrasound waves, thereby causing them to illuminate the tissue or area they are in. Gases used in microbubbles include, but are not limited to, air, nitrogen, argon, or perfluorocarbon. Microbubbles may also have different size regimes, for example, from 1-1,000 μm in diameter.

Microbubbles serve as effective UCA's, where their echogenicity is dependent on variables such as the gas that is included, the size of the bubble, the coating of the bubble shell, and how many bubbles are injected or implanted. However, the life-span of the microbubbles' echogenicity is significantly limited, that is, the length of time they are ultrasound visible before the bubbles dissolve, rupture, burst, or any other phenomenon that may yield the bubbles non-echogenic. Furthermore, ultrasound itself may be the cause of rendering the microbubbles non-echogenic. There are applications where ultrasound-mediated destruction of microbubbles is useful such as for thrombolysis or drug delivery. It is understood that high-intensity ultrasound causes soft-shell microbubbles to rupture. Hard-shell microbubbles are generally preferred for applications where high-intensity ultrasound imaging is necessary and they do provide longer in vivo stability than soft-shell microbubbles.

One of the major problems in the field is the limited lifetime of the echogenicity of the microbubbles; usually, the echogenicity is on the scale of minutes to a few hours. This is often due to the air/liquid surface tension, resulting in diffusion of the gas out of the bubbles. The inclusion of perfluorocarbon (PFC) gas into microbubbles allowed microbubbles to have a lifetime of several minutes in vivo. Silica bubbles, one of the hardest shell microbubbles, have only been shown to be observed in vivo 8 hours after injection. There have been reports of microbubbles lasting greater than 1 year, which used a combination of surfactants as the shell (specifically, glucose syrup and sucrose stearate). However, these findings were in vitro, where the bubbles were contained in a viscous continuous phase and imaged with transmission electron microscopy (TEM), rather than ultrasound. As such, the bubbles were not exposed to any ultrasound waves that would cause them to dissolve or rupture quicker, which would be the case in vivo. These relatively short lifetimes have limited the translation of these agents into other biomedical applications that require ultrasound imaging for longer periods of time.

An additional major problem in the field is the lack of systems and methods capable of detecting implants imaged via ultrasonography and further analyzing them. These are especially useful in situations where the implant requires imaging and analysis beyond the implantation procedure itself; for example, to detect the implant's mechanical properties, location, safety within the body, and degradation over time. By coupling implant compositions that possess long-term echogenicity with systems and methods for analyzing ultrasound images of the implant, this provides a powerful tool for clinicians to track implants over long periods of time and offer patients a more reliable diagnosis.

### SUMMARY OF THE INVENTION

Embodiments of the present invention provide systems and methods capable of automatically detecting and qualitatively and quantitatively analyzing bodily implants in medical images, such as ultrasound images. Along with having clinical application due to an ability to detect disease-indicating abnormalities, the systems and methods taught herein provide data that can be used in medical device research and development as well as in clinical applications as a diagnostic or therapeutic method.

Bodily implants are typically engineered medical devices consisting of non-biological or biological materials. Previous work has shown that implants may be made more visible across the imaging modalities by adding contrast agents, such as microbubbles or radioactive dye. Regardless of the homogeneity of the contrast agent throughout the implant (which can impact the visible texture of the implant in medical media), the systems and methods of the present invention include one or more algorithm(s) which are capable of extracting Regions of Interest (ROIs) with or without increased visibility as a result of said contrast agents. In one embodiment, the one or more algorithms are capable of extracting and analyzing the ROI based on morphological features, which is computationally more efficient than previously existing methods. Advantageously, the one or more algorithms require no additional, manual input for such analysis. Furthermore, machine learning algorithms may be applied to identify and analyze the implants more accurately and efficiently, especially if algorithms are used more frequently or the number of images inputted into the algorithm are increased.

The systems and methods are capable of performing automated image recognition and analysis, specifically in the area of medical implants. Such systems and methods have one or more applications including, among other things, research and development, medical device engineering, quality assurance and quality control, as well as clinical applications such as therapeutic, diagnostic and/or check-up methods. Further, the systems and methods can also provide an examination method for patients who received a medical implant, which offers the ability for healthcare providers to provide patients with more meaningful and accurate diagnoses and treatments. For example, if an implant is capable of degradation or reversal, then imaging must be performed to locate the implant prior to reversal. As the systems and methods are capable of determining the location of the implant, length of the implant, and other features, they are capable of guiding the degradation or reversal process and ascertaining its progress. Other uses and advantages of the systems and methods, only some of which are discussed herein, include quantification and/or qualification of one or more implant features including: echogenicity, length/width of the implant, the location of the implant, the homogeneity or heterogeneity of the implant, the degradation of the implant over time, and safety readouts such as fibrosis, immunological responses/tissue reactivity, and/or intracutaneous thickness.

Echogenicity is a measure of the brightness of the material on ultrasound and is an important measurement that can inform medical health professionals about drug delivery/release, sustained released, release of the contrast agents from the implant, or degradation of the implant itself. The ability to measure the length/width of the implant is important for cases where the length and/or width dictate efficacy or safety for the patient. Some implants may need to be adjusted or designed to fit with the patient's anatomical measurements. Other implants may degrade over time, and as such, their length/width may change. The following system and methods provide the healthcare professionals to confirm if the implant has the right size (i.e. length/width) within the anatomy of interest. The ability to measure the location of the implant is highly important, especially in anatomical areas where the implant may be able to migrate. The following system and methods provide tools for healthcare professionals to automatically locate and isolate the implant within the anatomy of interest to confirm it is in the correct location. Finally, the following system and methods may also identify and analyze various anatomical areas, in addition to the implant, to determine safety parameters such as fibrosis, inflammation, sensitization, or any other immunological response around the implant.

The systems and methods may also be utilized for guiding the reversal or degradation process of the implant. For example, the algorithms may detect a needle or catheter that enters the area of anatomical interest to deliver a stimulus (i.e. chemical or mechanical) to degrade the implant. Furthermore, the algorithms may track the degradation of the implant over time such as by analyzing the implant's length, width, homogeneity, and/or echogenicity, followed by providing confirmation for healthcare providers whether the implant was successfully removed, reversed, or degraded.

With respect to ultrasound, in particular, there is a significant need in the field for soft materials/implants that can be imaged with ultrasound, where the echogenicity lasts as long as the implant itself. The present invention expands the lifetime of ultrasound contrast agents (UCAs) beyond the span of tens of minutes or hours in vivo. Classic microbubble lifetimes are constrained by diffusion of the gas from the shell of the bubble. The present invention circumvents that problem by using UCAs that do not contain gas, but are still highly echogenic. This new type of UCA can be included in soft materials, whether injected or implanted, to render them echogenic for long periods of time. In embodiments, the material/implant can retain its echogenicity in whole or part, or in some cases the echogenicity of the material/implant may increase over time. For example, after a period of time, such as 3 months, 6 months, 9 months, 1 year, 1.5 years, 2 years, 5 years etc., the material/implant can retain 99% of its echogenicity, or retain 98%, or retain 90-97%, or retain 80-89%, or retain 70-79%, or retain 60-69%, or retain 50-59%, or retain 40-49%, or retain 30-39%, or retain 20-29%, or retain 10-19%, or retain 5-25%, or retain more than 5%, or retain more than 10%, or retain above 0% to 15% of its echogenicity.

For example, if implanting a soft material into a bodily duct, such as the vas deferens for purposes of male contraception, it is desired that the material will last a long period of time (i.e. >1 year). It would be useful for physicians to quickly and safely visualize the implant using ultrasound to ensure: a) it is still present, b) its location, c) its length and/or width, d) the implant's homogeneity, e) if the implant is degrading over time, f) how quickly the implant is degrading (if at all), and g) if there is any tissue reactivity around the implant including, but not limited to, fibrosis. Often, ultrasound imaging/confirmation may be required every few months i.e. 3 months, 6 months, 12 months, etc. As such, it is necessary that the soft material maintains its echogenicity beyond the initial ultrasound scan, seconds or minutes after implantation. This would also be applicable in any bodily duct, such as the fallopian tubes for female contraception, aneurysms, drug-delivery depots, drug delivery of small molecules, drug delivery of chemotherapeutics, drug delivery of protein cargo, drug delivery of peptide, drug delivery of oligonucleotides, void fillers after tumor removal, implants within the intramuscular space, implants within the subcutaneous space, implants used to space organs at risk during brachytherapy, implants between tissues spaces such as that found at joints, implants between bone and soft tissues, and other tissues, organs, and interstitial spaces. In embodiments, the implantable soft material with long-lasting echogenicity can be present in a bodily duct, lumen, organ or space that comprises one or more of an artery, vein, capillary, lymphatic vessel, a vas deferens, epididymis, or a fallopian tube; a duct, a bile duct, a hepatic duct, a cystic duct, a pancreatic duct, or a parotid duct; an organ, a uterus, testis, prostate, or any organ of the gastrointestinal tract or circulatory system or respiratory system or nervous system; a subcutaneous space; or an interstitial space.

The lifetime of the implant can be tuned from days to years, such as 1 year, 2 years, 3 years, and so on. By themselves, soft material implants are often non-echogenic, especially if their material properties are similar to the tissue around them. Previously, it has been shown that microbubbles may be included into vas-occlusive devices to enhance their echogenic properties, such as in US20170136143A1, which is incorporated by reference herein in its entirety. However, there are significant challenges that limited the commercialization of these microbubble-encapsulated devices. The first and foremost, is their echogenicity was limited from minutes to hours, depending on how many bubbles were included in the composition and how much of the implant was formed. This was due to the microbubbles dissolving or rupturing, or escaping the implant itself. Second, the microbubbles varied in size and homogeneity. As a result, some areas of the implant were significantly more echogenic than other areas of the implant. This could result in false readings for physicians who are trying to determine the size/length of the implant or if the implant is degrading over time. Finally, if the microbubbles escape the implant, there may be concerns around biodistribution.

Such advantages and applications, as well as disclosure for enabling reproduction of the systems and methods, are provided in the foregoing Detailed Description.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIGS. 1A-1D are images used to demonstrate an alternative path to obtain a binary image. FIGS. 1A-1D show that a gradient extracts a more complete gel than forming a binary image from the original image. FIG. 1A is the original ultrasound image of a hydrogel implant. FIG. 1B is the binary image that is obtained directly from FIG. 1A. FIG. 1D is the binary image obtained by using FIG. 1C; the gradient direction of FIG. 1A, as an intermediate step directly from FIG. 1C. FIG. 1D represents a more continuous representation of the hydrogel than FIG. 1B.

FIGS. 2A-2C are ultrasound images showing ROI segmentation of a hydrogel implant with added materials for echogenicity.

FIG. 3 is a plot profile of pixel distance versus intensity across the bounding box of an ROI.

FIG. 4 is a graph displaying the correlation between automated and manual ROI extraction and analysis. The high R² value shows that 96.5% of the automated data aligns with the manual measurements.

FIGS. 5A-C are ultrasound images showing alternative examples of hydrogel ROI extraction.

FIG. 6 is a flowchart exemplifying a specific embodiment of the method.

FIGS. 7A and 7B are two different ultrasound images showing extraction and segmentation of the vas deferens.

FIGS. 8A-C are ultrasound images showing a cascade object detector machine learning algorithm attempting to extract hydrogel implants from the images.

FIG. 9 is a graph displaying the average plot intensity (echogenicity) of a soft material containing no contrast agents over time.

FIG. 10 is a graph displaying the average plot intensity (echogenicity) of polystyrene microbubbles over time.

FIGS. 11A and 11B are an ultrasound image of a soft material containing a carbon-based nanomaterial/allotrope.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Described herein are systems and methods capable of automatically and semi-automatically identifying, extracting, and analyzing bodily implants from medical imaging media. Medical imaging media can refer to, but is not limited to, ultrasound, x-ray, MRI, CT scans, and PET scans. The retrieved image can be or include a 2D image, a 3D image, a pre-recorded video, a live video, or a stack/set of images/videos. The environment in which the medical media is retrieved can be either in vitro, ex vivo, or in vivo. Ex vivo representations can include organs, ducts, vessels, or tissues excised from the body and used in testing, or bodily simulations (such as ultrasound phantoms). For instance, gelatin ultrasound phantoms are often used to mimic soft tissue. In vivo may refer to the targeted areas of an implant, including but not limited to bodily lumens, tissues, organs, ducts, and interstitial tissues. Areas in the body that can be the site of a medical implant include, but are not limited to, vas deferens, fallopian tube, aneurysms, urethra, ureters, arteries, veins, lungs, kidneys, gastrointestinal organs/tract, breasts, and the heart, and combinations thereof.

The implant or implantation comprising the soft material with a selected level of echogenicity, and/or that is the target of the imaging systems and methods can include, but are not limited to, hydrogels, coatings, microparticles, microgels, nanoparticles, nanogels, foams, sponges, electrospun meshes or fibers, microfibers, and nanofibers, and combinations thereof. Such materials can comprise one or more polymers, including one or more of natural or synthetic monomers, polymers or copolymers, biocompatible monomers, polymers or copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers such as polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly(vinylmethylether), poly(ethylene oxide), poly(ethyl (hydroxyethyl) cellulose), polyoxazoline (POx), wherein x is a number from 1-5, polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethylene glycol (PEG) or any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), Nisopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or poly(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetrafluoroethylene RFE such as TEFLON® RFE or KRYTOX® RFE, fluorinated polyethylene (FLPE or NALGENE®), methyl palmitate, temperature responsive polymers such as poly(N-isopropylacrylamide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lactide-co-caprolactone (PLCL), and/or chitosan, and combinations thereof.

Automated Image Recognition of Medical Implants

The analysis of the implants according to the systems and methods taught herein provide data on the quantified intensity (such as echogenicity) and homogeneity of the implant. Other data which the systems and methods are capable of providing can include, but is not limited to, density, size, deformation, formation time, formation efficacy and stability, degradation of the implant, and/or confirmation of the implant's presence or lack thereof. Furthermore, relationships determined over a set of images provide correlations and/or mathematical relationships between brightness, area, degradation, longevity, and/or the material or characteristics of the material of the implant.

The following provides exemplary embodiments of methods; however, as will be recognized by one skilled in the art, the specifics of the algorithms disclosed herein can include any alternative order of steps and/or different parameters depending on the image type, quality, and/or goal.

Morphological Analysis

According to one embodiment, an algorithm extracts the ROI via a number of image recognition and image segmentation techniques. Following formation of a binary image, the edges are determined and distinguished, for example using the ‘Sobel’ method. Alternatively, the ‘Prewitt’, ‘Roberts’, ‘Log’, ‘Canny’, or other alternative edge-detection algorithms can be used. Once the edge information is extracted, numerous morphological techniques may be applied to the image(s). These include, but are not limited to erosion, dilation, and filling techniques, which formulate and extract ROIs. In one aspect, the ROI is defined by the largest ROI, or a combination of multiple ROIs, defined as clustering. The entire ROI, regardless of visual homogeneity, can be accurately extracted based on a series of tests that evaluate the probability of likely candidates. For instance, distance-based clustering is an algorithm/test that is successful in extracting only the appropriate ROI candidates. The extent of technique application may depend on the image itself. For instance, noisier images may require more erosion and filtration of the image prior to analysis and extraction.

Following the above methods of erosion, dilation, filling, and/or clustering, the algorithm forms a binary image that contains only the ROIs associated with the implant itself. Using this extracted image, the algorithm can obtain the location, boundaries, and size of the final extracted implant image. A bounding box encompassing the location of the remaining ROIs provides parameters to be used in the rest of the algorithm. Alternatively, the parameters can be manually provided if the user prefers. This shape can be, but is not limited to, a box, polygon, circle, or blob. These parameters are used to determine echogenicity (via an intensity calculation) and homogeneity (via a plot profile) of the implant.

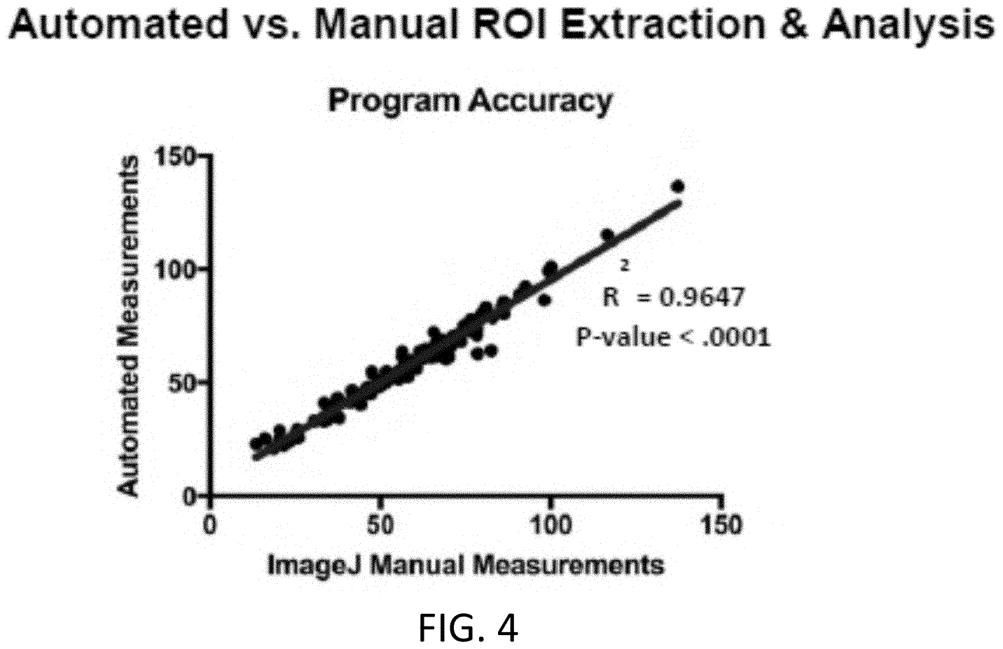

Echogenicity is defined as the average intensity, or brightness, of the ROI within the image. This is achieved by averaging the original pixel values across the extremities of the binary ROI boundary locations within the bounding box. The brighter the ROI is, the more echogenic that particular implant formulation is. If no boundary exists, the extremities of the adjacent rows within the bounding box are used. For example, echogenicity can be determined by imaging one or more regions of interest (ROI) and measuring the mean gray level within one or more of the selected regions of interest (ROI). The image is analyzed, e.g., using the software and/or algorithms of the invention, on a pixel-by-pixel basis to assign one of 256 gray-scale values to one or more pixels in the ROI (the values ranging from 0 (black) to 255 (white)). According to embodiments, the methods disclosed herein are capable of detecting the gray-scale value of one or more or each pixel in the ROI of the image. Then a mean gray-scale value is determined for the ROI by adding up the individual gray scale values assigned to the pixels and dividing by the number of pixels in that ROI, thus providing a gray-scale mean (GSM) to represent the mean gray-tone frequency distribution of the pixels included in the ROI. The GSM can thus be used as a quantitative measure of echogenicity of selected regions of interest. See, e.g., Mayans, D.; Cartwright, M S.; Walker, F.O. Neuromuscular ultrasonography: Quantifying muscle and nerve measurements. Phys. Med. Rehabil. Clin. N. Am. 2011, 23, 133-148. In embodiments, the gray-scale mean for the ROI corresponds with the level of echogenicity for the compositions and/or implants that are the subject of the ROI. For example, the gray-scale value for the compositions and/or implants can range from above 0 to 255, such as from 5-250, or from 10-240, or from 15-230, or from 20-220, or from 30-210, or from 40-200, or from 50-190, or from 60-180, or from 70-170, or from 80-160, or from 90-150, or from 100-140, or from 110-130, or from 120-125, or from above 0 to 150, or from 15-100, or from 25-125, or from 35-185, or from 45-195, or from 55-225, or from 65-235, or from 75-255, or from 30-220, or from 50-180, or from 40-215, or any ranges in between including any of these values as starting and/or endpoints of the range. A plot profile is a graph depicting pixel distance versus grayscale-value (see FIG. 4). Greater consistency across the graph implies that the ROI is homogenous while inconstancy implies inhomogeneity. This can be important in research aspects when various implant formulations are being tested for diffusion of various additives. For instance, a user may find that one additive (i.e. ultrasound contrast agent) diffuses more evenly in a polymer than another, potentially implicating such additive as the ideal additive. Another example is this method can be used to determine which type of mixing (i.e. sonication or vortexing) is ideal for suspending an additive within a formulation. In a clinical setting, this application may be used for informing the physician that a consistent implant was formed or gelled in the case of a hydrogel. An alternative method of measuring homogeneity could be from a histogram or gray level distribution analysis.

Image Gradient

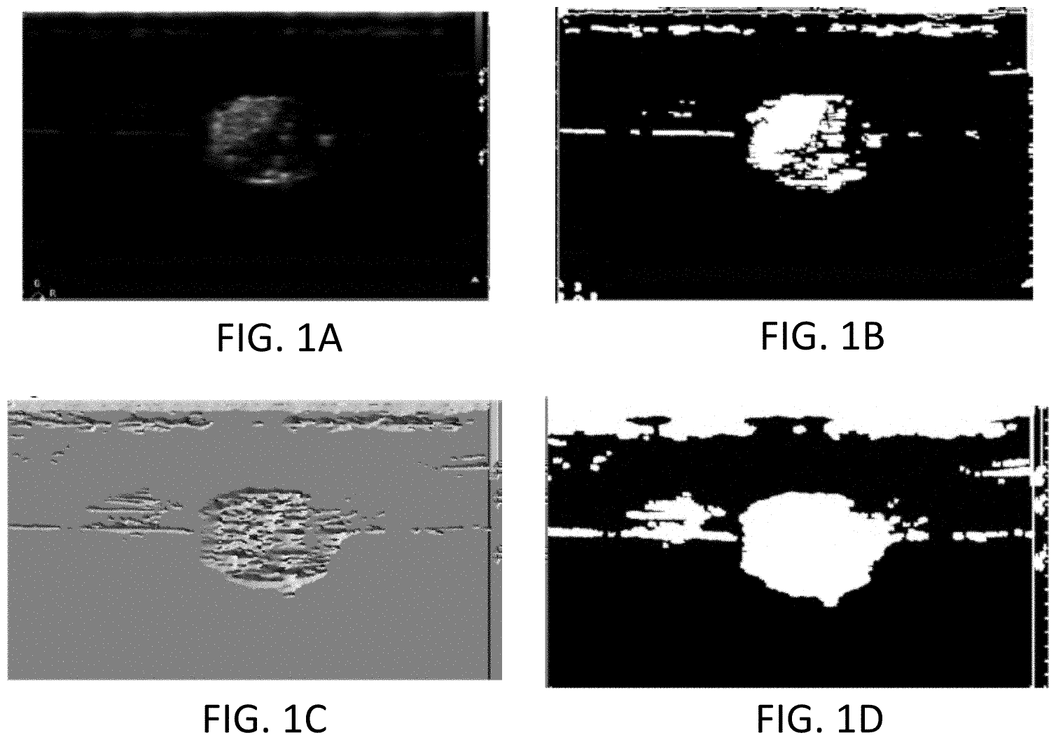

The gradient of the image can be calculated and used to develop an alternative binary image. This can be done by calling gradient function in the programming language of the user's choice. Alternatively, convolution kernels such as Sobel filters may be used to form a gradient image by measuring intensity changes and corresponding directions. Following the formation of the gradient image, one can convert to a binary image. Subsequently, the methods used above in “Morphological Analysis” may be used to extract and analyze the ROI. Such methods include eroding, dilating, and/or clustering. In one aspect, this method is ideal for implants with a heterogeneous, disjointed appearance (see FIGS. 1A-1D).

Machine Learning

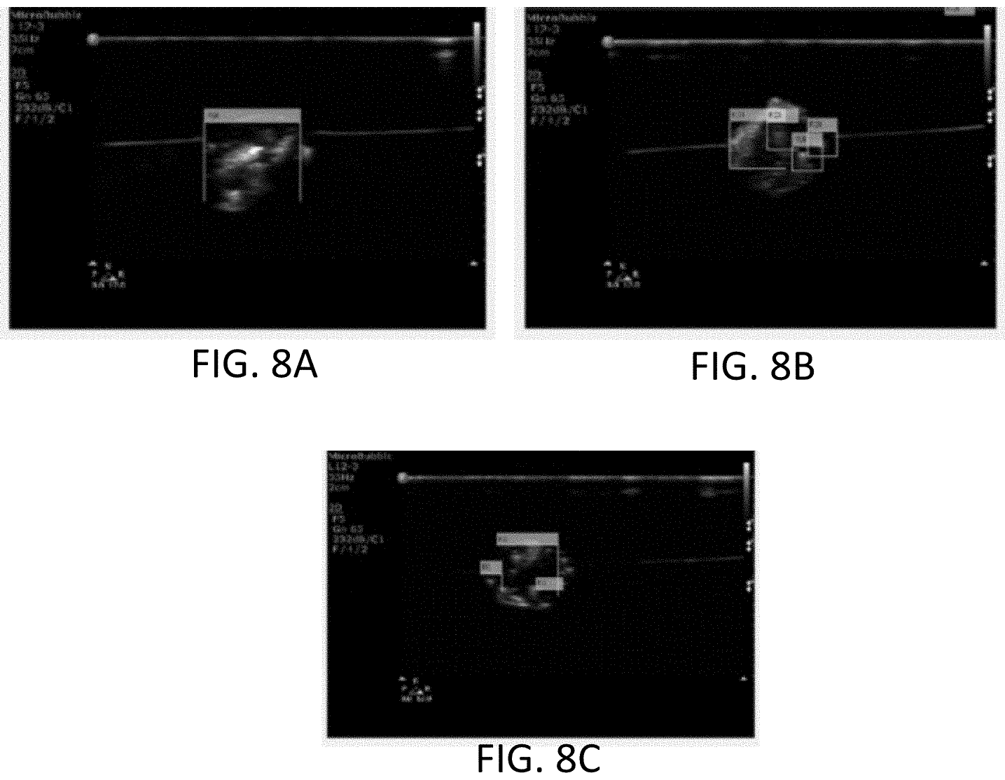

Machine learning can be used either as the solution to ROI extraction or in conjunction with alternative algorithms. In one aspect, machine learning provides the parameters of an ROI for the algorithm described in the “Morphological Analysis” section above. Alternatively, a classification network can be developed as a replacement to clustering to see which ROIs are associated with the true implant. In this case, an array of ROIs would be inputted and given a classification of ‘yes’ or ‘no’, depending on whether or not the ROI is part of the implant. Alternatively, machine learning can be used to accomplish the entire task, including, but not limited to, ROI extraction and data quantification. Examples of machine learning include, but are not limited to, deep learning and neural networks (e.g., convolutional neural networks). These will learn features of the body implants in a given environment and provide automatic extraction with or without external image manipulations. The use of machine learning has been taught to locate and form bounding boxes around ROIs. The present systems and methods also teach using a modified algorithm for implant detection or further analysis. As described herein, a cascade object detector can be trained to recognize ultrasound images of implants (e.g., precipitates or hydrogels). The results indicate that machine learning is successful in automatically extracting the implant from ultrasound images (see FIG. 8A-C). It has also been shown that additional data (e.g. images) will improve the accuracy of the algorithm.

Feature-Specific Extraction

Feature-specific extraction will depend on the type of feature being searched for. In one example, Hough transforms can be used to either extract or remove lines within an image. Hough transforms, along with being able to be adapted to detect geometric shape, are often used to detect points that form lines. When points on a line are graphed on the planes of their constant, they will form two intersecting lines. When multiple lines intersect at the same point in the alternative plane, this indicates the points form a line in the original plane. Similarly, regression analysis can be used to filter out the outliers in an image. For example, the location and/or value of every pixel in an image can be plotted to determine the residuals against the mean. High residuals can be excluded from the final image as a way of reducing image noise as they deviate too greatly from the mean. Similarly, a histogram can be created of said pixels as an alternative to clustering. Small bins within the histogram are not likely to be a part of the implant. Alternatively, feature-specific filters can be created to iterate over the image. Such filters will search for specific qualities such as lines, blobs, or corners. This can be used for either extraction or removal of the feature at hand. Alternatively, if a block of noise is common amongst a large number of images, a function can be extracted from a portion of that image and used to remove chunks of noise.

Shape Specific Extraction

The ROI can also be extracted by having the program search for a specific shape, or near specific shape, that the implant will adopt in situ. Examples of such shapes refer to both 2D and 3D aspects, including but not limited to circles, squares, rectangles, triangles, spheres, cubes, cylinders, or pyramids, or even irregular shapes. These shapes can be extracted via morphological techniques and shape-fitting, among other methods. For instance, imposition of a probable shape on the ROI can be used to extract probability maps for given features and their respective locations. Furthermore, these probability maps can be used alternatively in any of the alternative methods listed here (e.g., morphological techniques, machine learning, or level sets) Provided an ideal 3D shape, 2D images can be parameterized and reconstructed into a representative 3D shape, which would provide alternative data in a real-world aspect.

Multi-Image Analysis

If a sample of representative images is present, the images can be compared to each other for commonalities and differences. Such comparison provides information on extractable and removable features from the image at hand. For instance, bulk intensity can indicate common features between images. Furthermore, given a stack of images, a composite image can be used to form a single representative image that is used for all calculations and analysis. In one aspect, this is ideal for a heterogeneous implant that results in varying ultrasound images depending on the angle the user holds the probe for imaging.

Texture Analysis

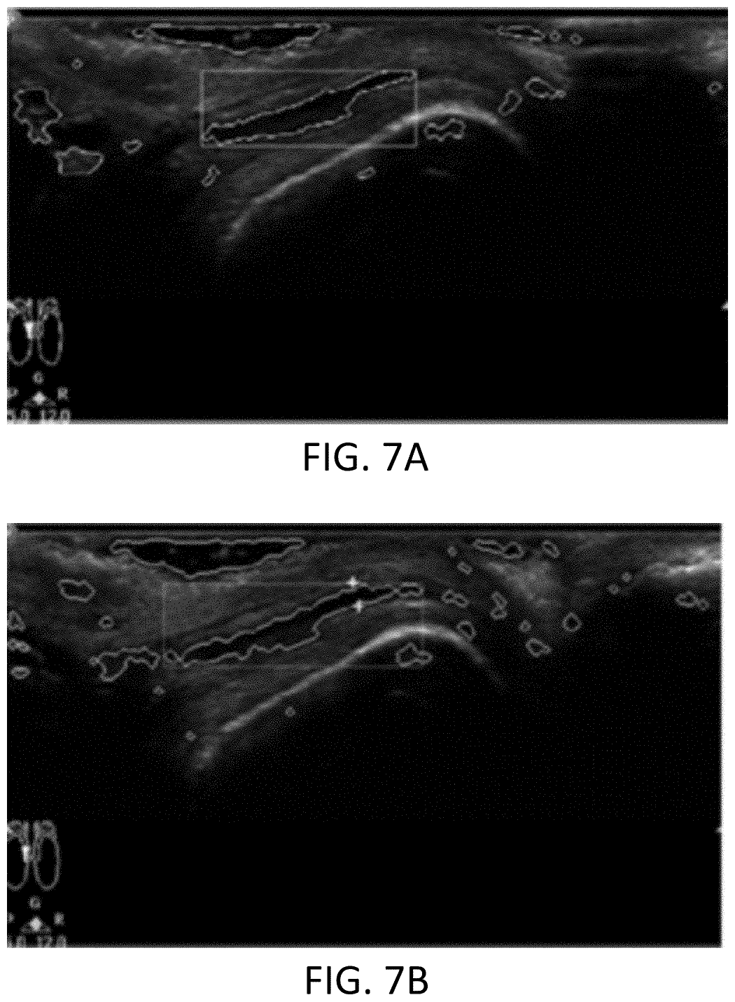

Texture analysis can be used to identify ROIs based on their location in the environment. For instance, in vivo implants may be recognized by the recognition of surrounding smooth muscle, such as smooth muscle contacting inorganic or non-biological materials. Alternative textures include epidermis, striated muscle, or lumens. The vas deferens, which has a lumen surrounded by thick smooth muscle, has been shown to be identified by such analysis (FIGS. 7A and 7B). Implants within ultrasound phantoms may be recognized by the texture of the phantom material or the implant itself. Texture analysis can compare the variation in pixel intensities. Little variation correlates to smooth textures while large variation may correlate to rough textures. These differences can be used to form region boundaries. In instances where the implant will have a different appearance than the phantom on the given imaging modality, texture analysis may be used to segment these regions. In the case of the vas deferens, texture analysis may be used to extract the implant from a tube in a gelatin phantom as well as the implant in the vas deferens itself. However, these same principles may be applied to implants within other bodily ducts, lumens, tissues, or organs.

Alternative Algorithms

Alternative algorithms to the methods above include, but are not limited to, template matching, level set segmentation, median filtering, and active contours. Level sets are capable of searching for the cross sections of an image. For instance, a vas deferens may have either a circle or rectangular cross section depending on the imaging angle. This makes level sets good candidates for extraction of the vessel. Similar algorithms may be used to detect blood vessels or tubes (e.g. fallopian tubes). Similarly, active contours attempt to impose an ideal shape on an image and iteratively adjust to match the true contours of said shape. Active contours are also a candidate for segmentation of vessels like the vas deferens because of its roughly rectangular or circular cross sections that provide a consistent starting shape. Active contours may also be used for heart implants, which tend to have a rounded shape. Alternatively, fibroadenomas are known to have a smooth surface and a well-defined shape. Using an elliptical shape as the initial curve of an active contour would be successful in locating a fibroadenoma in an ultrasound image. These methods are capable of extracting an implant in a medical image. Following extraction, the above descriptions of analysis (such as echogenicity and homogeneity) can be used to obtain further information. Median filters are a preferred method for removing noise while still maintaining the shapes of the edges. Since the morphological techniques described above require edge detection for success, median filtering could serve as a successful preprocessing step. An algorithm that is geared towards imaging bodily implants is applicable.

Image Capturing

As ultrasound technology becomes more advanced and devices become more portable, the types of machines and probes that can be used for image capturing has expanded. Examples of ultrasounds that may be used for capturing images of the implant include, but are not limited to, midrange ultrasound machines (e.g. Philips HD XE 11 or Philips Affiniti 50), tablets (e.g. eZono 4000), non-mobile handheld devices (e.g. Rivanna Accuro), and mobile-connected handheld devices (e.g. Butterfly iQ). Furthermore, the transducer probes may be varied depending on the type of ultrasound and application. Examples include, but are not limited to, linear, curvilinear, phased array, endovaginal, biplane, triplane, drop-in, T-shaped, and endocavity. The ultrasonic energy can be administered at a frequency between 1 and 20 MHz. The intensity of the energy can be between 0.1 and 1 Watts/cm2. Furthermore, the energy can be administered in a pulsed or continuous mode. In imaging a duct such as the vas deferens, a higher frequency probe such as the hockeystick probe or high-frequency linear produce the best resolution. While transducers are traditionally piezo crystal based, there are also advancements in “ultrasound-on-chip” technology where there are made on a single silicon chip. The ultrasound probe/device may also have needle guidance features, which is useful when a needle is used for inserting and/or reversing implants. In some cases, Doppler is helpful for identifying certain anatomical areas, such as differentiating the vas deferens from the testicular artery. The systems and methods described include algorithms that take data from Doppler imaging into account. Finally, the images captured may be any given angle ranging from axial to longitudinal.

Implementation

According to embodiments, the methods can be implemented in a set of computer-executable instructions (used interchangeably herein as “software”, “software application”, “program”, “software program”, and grammatical variations thereof) installed directly onto an imaging device (such as ultrasound) to provide efficient, in-house analysis on pre-captured and/or live imaging. In one aspect, live analysis is used for clinical applications and confirmation of correct product usage and function, such as whether an implant was successfully implanted for its intended purpose.

Alternatively, in situations where access to imaging technology is not ideal, the methods can be implemented as a software application for computers, handheld devices, or instruments including portable devices (e.g., a laptop computer, tablet, or cell phone). Any such computer, handheld device, or instrument can be configured through software to be used in conjunction with an imaging device, such as during image processing (e.g. in real time) within the imaging device itself, through image retrieval, and/or through images produced and then stored outside the imaging device such that one or more of the methods are performed. The software may also be built into the imaging device, where the image processing is performed on stored images at a later date or time than when the images were initially captured. The computer, handheld device, or instrument can include such hardware as one or more processors (CPU), a display, input/output ports, and a memory, as well as software such as an operating system and a Graphical User Interface (GUI). In other aspects, the methods can be used at the programming level in a developer's preferred computing environment (e.g., Matlab). One application of this method, by way of example, implemented in software allows for manual selection of the images to be analyzed (either by selection through a GUI or dialog boxes). Such software application can also enable the user to choose which measurements to output (e.g., average intensity and density). Furthermore, the software application can also ask the user which visual representations the user prefers (e.g., the original image, the extracted ROI, the outlined image, and/or the associated plot profile of said ROI).

In one embodiment, the software application or computer-executable instructions are capable of being stored on a memory storage of the computer, handheld device, or instrument and instructing one or more processors (CPU) of such computer, handheld device, or instrument to perform any of the methods, processes, operations, and algorithms described herein. The computer-readable instructions can be programmed in any suitable programming language, including JavaScript, C, C#, C++, Java, Python, Perl, Ruby, Swift, Visual Basic, and Objective C. The memory can be or include a non-transitory computer readable storage media such as RAM. Other components of the computing device can include a database stored on the non-transitory computer readable storage media. As used in the context of this specification, a non-transitory computer-readable medium (or media) may include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM.

Examples of Automated Image Recognition

The binary images obtained from various image channels are compared in FIGS. 1A-D. FIG. 1A is the original ultrasound image of a hydrogel implant. FIG. 1B is the binary image that is obtained directly from FIG. 1A (via edge detection, dilation, and filling). FIG. 1C is the gradient direction obtained directly from FIG. 1A. FIG. 1D is the binary image obtained directly from FIG. 1C. FIG. 1D, obtained by using the gradient as an intermediate step, provides a more continuous representation of the hydrogel than FIG. 1B.

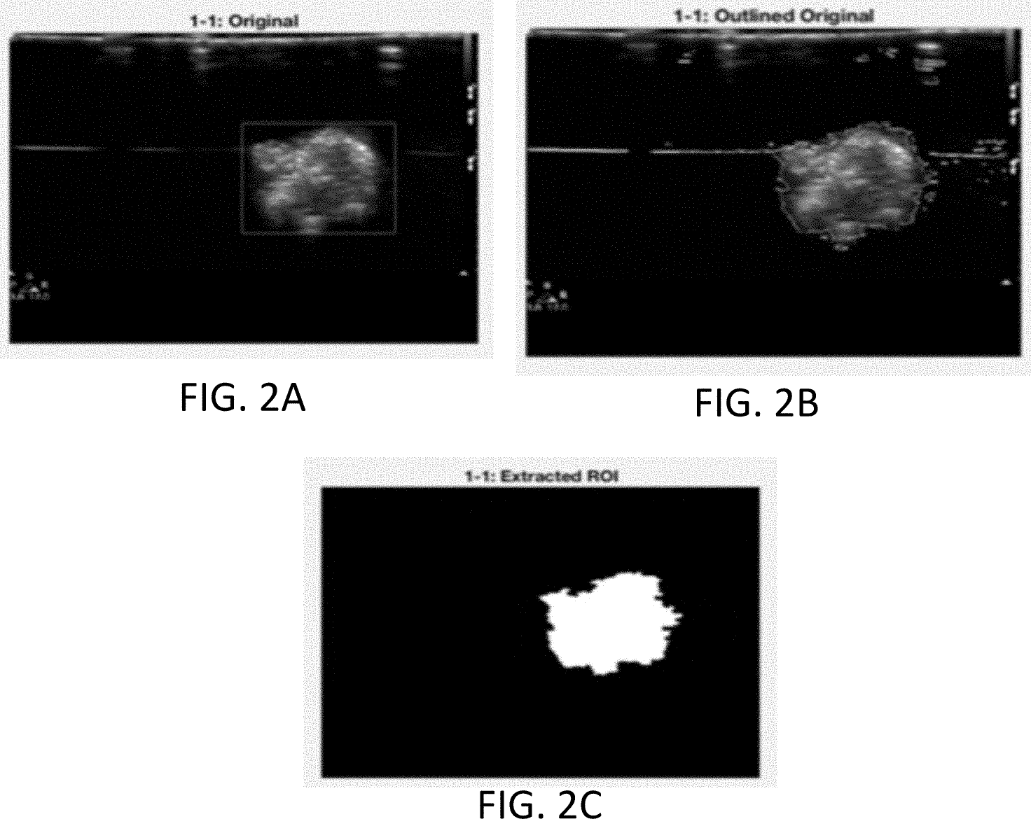

FIGS. 2A-2C show an example of ROI segmentation of a hydrogel implant with added materials for echogenicity. “1-1: Original” in FIG. 2A corresponds to the input image with a green box drawn around the ROI. The location of the box is determined by the software program automatically and is drawn as one form of program output. “1-1: Outlined Original” in FIG. 2B corresponds to the outlining of all ROI boundaries. “1-1: Extracted ROI” in FIG. 2C corresponds to the binary extraction of the entire ROI. A combination of all three images, as well as data used in image acquisition, is utilized to retrieve quantitative ROI data.

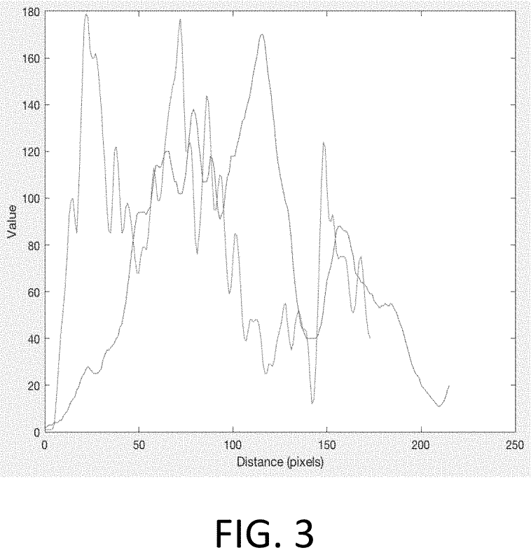

The plot profile in FIG. 3 represents pixel distance versus intensity across an ROI. The profile is calculated and plotted by taking 2D, linear cross sections of the extracted ROI. Such a plot profile may serve as a measure of homogeneity within the implant. For instance, if glass microspheres were added to a hydrogel to increase echogenicity, the plot profile provides a graphical representation of how well the microspheres were able to disperse within the implant. The homogeneity measurement provides valuable information in the research and development of said implant when determining the ideal volume or type of microspheres to add to the implant. For instance, gas microbubbles may disperse more evenly within a product than glass microspheres or chitosan. Such dispersal would make gas microbubbles one of the ideal contrast agent for ultrasound images where homogeneity is important. If the additive disperses evenly, the entire implant becomes echogenic. Alternatively, if the additive is unable to disperse evenly in the implant, the implant will have a disjointed appearance on the imaging modality, and only portions of the implant will be visible.

FIG. 4 shows a representation of accuracy of the inventive, automated algorithm when compared to Image) data, an industry accepted standard (Image) is a public domain, Java-based image processing program developed at the U.S. National Institutes of Health). To test the accuracy of the algorithm, Image) was used to provide a manual standard of comparison. The above plot shows the correlation between ImageJ's average intensity measurement and the fully-automated method of this Example. The two methods had a 0.965 R² value, which indicates a 96.5% accuracy in the quantitative measurements. Furthermore, the p-value indicates that the correlation is statistically significant. This indicates that the automated algorithm is sufficient to replace the manual extraction and analysis of the implant within various imaging modalities.

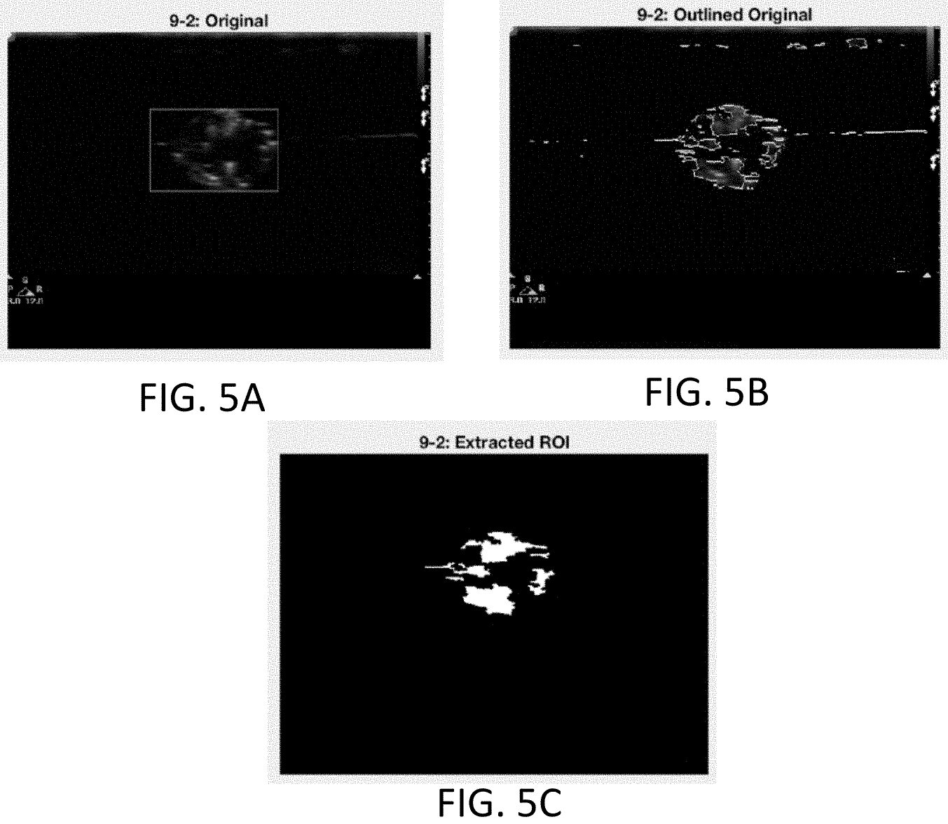

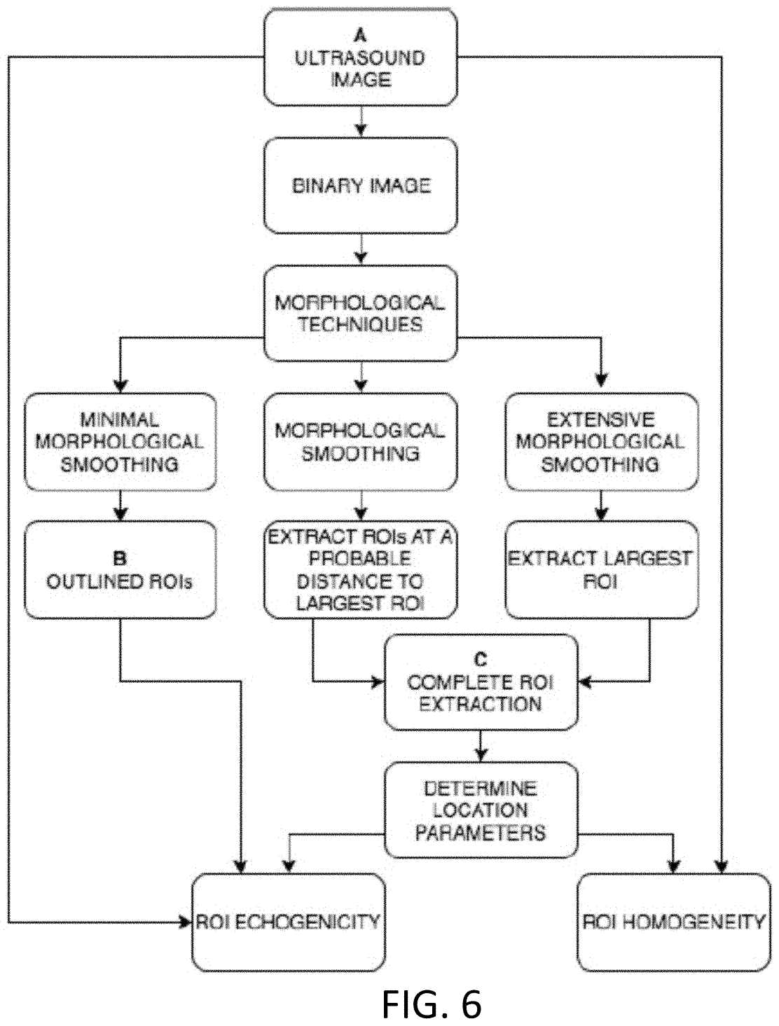

FIGS. 5A-5C and FIG. 6 exemplify an embodiment of the algorithm used specifically to image hydrogel implants with ultrasound. In particular, FIGS. 5A-5C demonstrate an alternative example of hydrogel ROI extraction. This specific implant is heterogeneous and presents as a disjointed ROI. The software program of this Example is able to successfully extract and analyze the entire ROI. The order and meaning of the images correspond to what is described in FIG. 2, but with an alternative hydrogel. R²=0.9647 p-value<0.0001. The flowchart in FIG. 6 shows a representation of one specific embodiment of the algorithm. This is used specifically to image hydrogel implants with ultrasound.

In this example, ultrasound images are used in the extraction and analysis of hydrogel implants. The method outputs data to represent the ROI's echogenicity and homogeneity (though numerous other measurements can be reported following the successful extraction of the ROI, labelled C on the flowchart). The correlation between the ultrasound images and the algorithm flowchart are as follows: “9-2: Original” corresponds to “A” (Ultrasound Imaging) (sans the green box), “9-2: Outlined Original” corresponds to “B” (Outlined ROIs), and “9-2: Extracted ROI” corresponds to “C” (Complete ROI Extraction) on the flowchart. The combined use of “A”, “B”, “C” and the respective data they provide allows for fast and accurate segmentation and analysis of the implants. The green bounding box is obtained from the boundaries of the final binary image in “C”. The outline imposed on “B” is used to determine the extremities of the implant within the parameters of the green bounding box. The pixel values within each of these extremities is then summed. The process continues iterating through every row within the bounding box. The average of the sum is taken and provides an average intensity. This value directly correlates to echogenicity. Alternatively, the homogeneity can be evaluated by plotting the distance versus pixel value across the bounding box.

FIGS. 7A and 7B show two examples of extraction and segmentation of the human vas deferens in two different ultrasound images, using the methods described herein. As such, it was feasible for the algorithm to automatically detect the organ or tissue of interest within human input. By detecting the inner lumen of the vas deferens, this software may be used to guide the implantation procedure for the physician. Furthermore, the algorithm could detect an implant within the vas deferens and analyze the implant's safety and efficacy over a period of time.

FIGS. 8A-8C show an example of the results from a cascade object detector machine learning algorithm that detected a hydrogel (containing minimal ultrasound contrast agents) without manual input. The machine learning algorithm selected the ROI for further analysis. As more data is collected, the algorithm can be continually trained and optimized for improved accuracy.

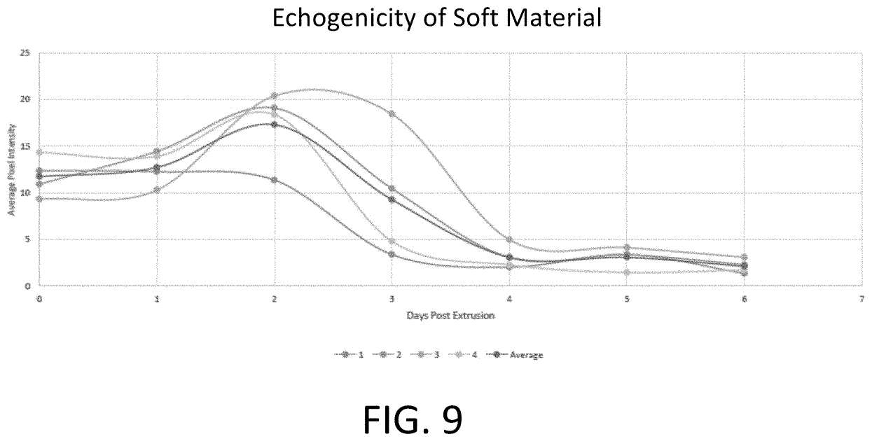

FIG. 9 demonstrates the need for an ultrasound contrast agent that has long-lasting echogenicity. In this example, the soft material does not contain any ultrasound contrast agents and is innately echogenic once injected/formed for 3 days post-extrusion. This is most likely due to the implant containing air bubbles formed by the extrusion from a needle. By 4 days, the implant is no longer echogenic (meaning ultrasound visible) even though the implant itself has not changed its size, structure, or durability. This is due to the unstable nature of the air bubbles trapped within the soft material.

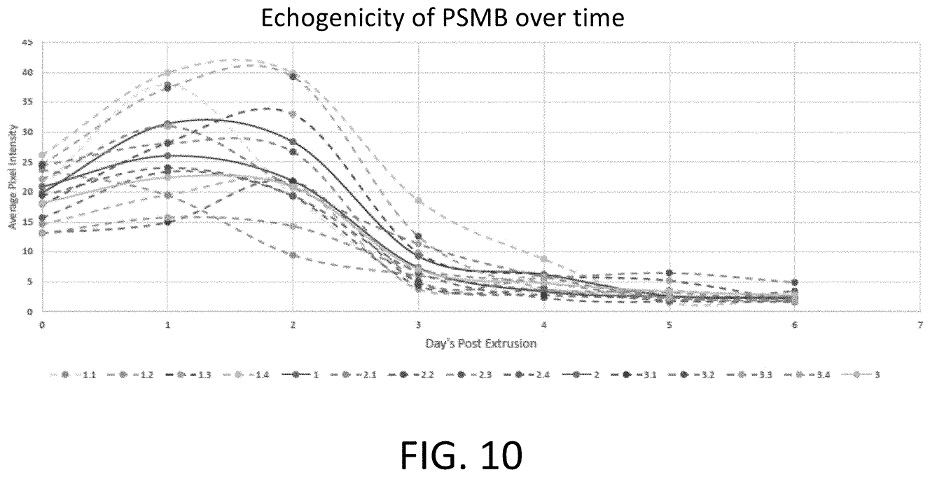

FIG. 10 also demonstrates the need for long-lasting ultrasound contrast agents. In this example, polystyrene microbubbles (PSMB) were synthesized and imaged over time. While the PSMB were highly echogenic for 2 days post-extrusion, by day 3, the bubbles ruptured and lost their echogenicity. As a result, these bubbles would not be clinically useful for rendering soft materials echogenic for long periods of time (e.g. months or years).

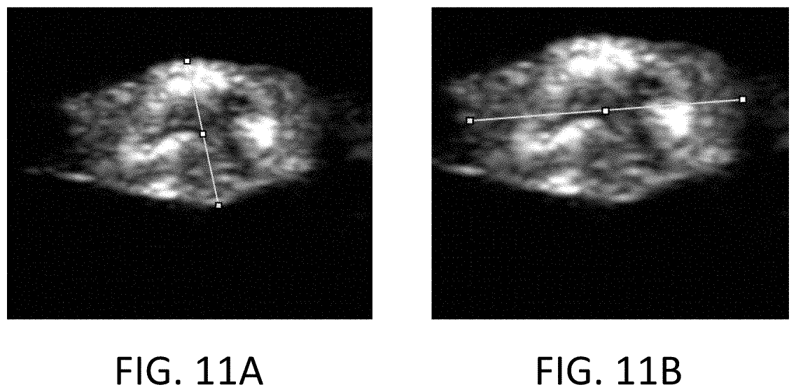

FIG. 11A and B are the same ultrasound image of a soft material (hydrogel) containing functionalized graphene nanoplatelets. The formed material is highly echogenic, is homogenous such that the perimeter of the material can be clearly identified and measured, and thus, the material is able to be detected using the systems and methods described herein. Furthermore, this composition is predicted to be highly echogenic for months or years after formation for the reasons described below.

Compositions with Long-Lasting Echogenicity

There is a significant need in the field for soft materials/implants that can be imaged with ultrasound, where the echogenicity lasts as long as the implant itself. The present invention expands the lifetime of ultrasound contrast agents (UCAs) beyond the span of tens of minutes or hours in vivo. Classic microbubble lifetimes are constrained by diffusion of the gas, which can be slowed using different surfactants on the surface of the bubble. The present invention circumvents that problem by using UCAs that do not contain gas, but are still highly echogenic. This new type of UCA can be included in soft materials, whether injected or implanted, to render them echogenic for long periods of time. In embodiments, the material/implant can retain its echogenicity in whole or part, or in some cases the echogenicity of the material/implant may increase over time. For example, after a period of time, such as 3 months, 6 months, 9 months, 1 year, 1.5 years, 2 years, 5 years etc., the material/implant can retain 99% of its echogenicity, or retain 98%, or retain 90-97%, or retain 80-89%, or retain 70-79%, or retain 60-69%, or retain 50-59%, or retain 40-49%, or retain 30-39%, or retain 20-29%, or retain 10-19%, or retain 5-25%, or retain more than 5%, or retain more than 10%, or retain above 0% to 15% of its echogenicity.

For example, if implanting a soft material into a bodily duct, such as the vas deferens for purposes of male contraception, it is desired that the material will last a long period of time (i.e. >1 year). It would be useful for physicians to quickly and safely visualize the implant using ultrasound to ensure: a) it is still present, b) its location, c) its length and/or width, d) the implant's homogeneity, e) if the implant is degrading over time, f) how quickly the implant is degrading (if at all), and g) if there is any tissue reactivity around the implant including, but not limited to, fibrosis. Often, ultrasound imaging/confirmation may be required every few months i.e. 3 months, 6 months, 12 months, etc. As such, it is necessary that the soft material maintains its echogenicity beyond the initial ultrasound scan, seconds or minutes after implantation. This would also be applicable in any bodily duct, such as the fallopian tubes for female contraception, aneurysms, drug-delivery depots, drug delivery of small molecules, drug delivery of chemotherapeutics, drug delivery of protein cargo, drug delivery of peptide, drug delivery of oligonucleotides, void fillers after tumor removal, implants within the intramuscular space, implants within the subcutaneous space, implants between tissues spaces such as that found at joints, implants between bone and soft tissues, and other tissues, organs, and interstitial spaces. In embodiments, the implantable soft material with long-lasting echogenicity can be present in a bodily duct, lumen, organ or space that comprises one or more of an artery, vein, capillary, lymphatic vessel, a vas deferens, epididymis, or a fallopian tube; a duct, a bile duct, a hepatic duct, a cystic duct, a pancreatic duct, or a parotid duct; an organ, a uterus, testis, prostate, or any organ of the gastrointestinal tract or circulatory system or respiratory system or nervous system; a subcutaneous space; or an interstitial space.

Soft materials are frequently used in a variety of biomedical, tissue engineering and drug delivery applications. Soft materials may include, but are not limited to, hydrogels, coatings, microparticles, microgels, nanoparticles, nanogels, foams, sponges, electrospun meshes or fibers, microfibers, and nanofibers, and subsequent combinations. These materials can be composed of polymers which include, but are not limited to polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers such as polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N isopropylacrylamine) (PNIPAM), poly (vinylmethylether), poly (ethylene oxide), poly (ethyl (hydroxyethyl) cellulose), polyoxazoline (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethylene glycol (PEG) or any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or poly(L-lactic acid)), poly(D,L-lactic acid), poly(D,L-lactide), poly dim ethyl siloxane or dimethicone (PDMS), poly(isopropyl acrylate) (PIPA), polyethylene vinyl acetate (PEVA), PEG styrene, polytetrafluoroethylene RFE such as TEFLON® RFE or KRYTOX® RFE, fluorinated polyethylene (FLPE or NALGENE®), methyl palmitate, temperature responsive polymers such as poly(N-isopropylacrylamide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lactide-co-caprolactone (PLCL), and/or chitosan, and combinations thereof. Additionally, these polymers can exist as random copolymers and/or block co-polymers.

The lifetime of the implant can be tuned from days to years, such as 1 year, 2 years, 3 years, and so on. By themselves, soft material implants are often non-echogenic, especially if their material properties are similar to the tissue around them. Previously, it has been shown that microbubbles may be included into vas-occlusive devices to enhance their echogenic properties. However, there are significant challenges that limited the commercialization of these microbubble-encapsulated devices. The first and foremost, is their echogenicity was limited from minutes to hours, depending on how many bubbles were included and how much of the implant was formed. This was due to the microbubbles dissolving or rupturing, or escaping the implant itself. Second, the microbubbles varied in size and homogeneity. As a result, some areas of the implant were significantly more echogenic than other areas of the implant. This could result in false readings for physicians who are trying to determine the size/length of the implant or if the implant is degrading over time. Finally, if the microbubbles escape the implant, there may be concerns around biodistribution.

This present invention utilizes carbon allotropes as UCA's such that the soft material is rendered echogenic for long periods of time. The echogenicity of these carbon-based materials is not due to gas encapsulation, but is rather due to their unique structural properties. The echogenicity of these materials does not decrease over time due to the gas-liquid interface or after exposing the materials to ultrasound waves. However, in vivo applications of carbon allotropes are limited due to aggregation issues. This invention utilizes different approaches that stabilize the individual structures and prevent aggregation, specifically as they relate to soft materials. The result are materials with echogenicity with lifetimes greater than tens of minutes or hours.

By tuning the lifetime of the materials, the echogenicity of the embedded carbon allotropes for the lifetime of the implanted material may be tuned up to years. This is an important feature to allow continued visualization of implanted material within soft tissue past the time scale of the implantation of the material. The applications stand to expand the scope the ultrasound-based techniques for different applications. For examples, continued monitoring and confirmation of an implanted soft material within tissue provide mechanical support or fill a biopsy void or act as occlusions for cells, liquids, or other material. Another example would be a material that is acting as a spacer between tissue or as a drug deliver depot.

Echogenicity of tissue is derived from the echogenic property of that tissue versus those of the tissues around it. Incorporation of carbon allotropes into implantable materials at the nanometer and micrometer scale alters the subsequent properties of the materials and therefore may allow it to have different echogenic properties than that of the surrounding tissues. Coupled with extended lifetimes of the implant a long lived echogenic implant is generated.

The echogenicity of the soft material may be dependent on the type of carbon allotrope that is included in the composition, its thickness (i.e. number of sheets), size, concentration, functionalization, spacing, association with soft material, or interaction with soft material. Similarly, the mechanical and chemical properties of the soft material may be affected by the inclusion of carbon allotropes (as shown by Eisenfrats in US20180028715A1), which is incorporated by reference herein in its entirety. Mechanical properties can result in differences in echogenicity as seen with bone and adipose tissue for example. Additionally, the chemical composition and functionality such as containing acids, bases, metal chelators, protein ligands, cellular ligands, and polysaccharides may alter the local and global environment such that differences in echogenicity are observed.

Carbon allotropes include, but are not limited to, graphene, graphene powder, graphene oxide, nanoscale graphene oxide, reduced graphene oxide, graphene nanoribbons, graphene nanotubes, graphene sheets, graphene films, granulated graphene, graphene quantum dots, graphene nanoribbons, graphene nanocoils, graphene aerogels, graphene nanoplatelets, or any other carbon-based material or nanomaterial including but not limited to carbon nanotubes (single walled, double walled, or multiwalled), nanosheets, nanocones, nanoribbons, buckyballs, fullerenes, and the like, as well as combinations of any of these. These carbon allotropes may be included in the soft material prior to injection or implantation.

The type of carbon material or allotrope that is used may directly impact the echogenicity of the compound as an ultrasound contrast agent, and therefore, the echogenicity of the soft material. For example, the inclusion of multi-walled carbon nanotubes into a soft material may render the soft material more echogenic than if graphene oxide was included in the soft material.

The amount of the carbon allotrope used may directly impact the echogenicity. Inclusion of more allotropes may increase the echogenicity. Tuning of the echogenicity such that the material is differentiable from neighboring tissues is a key trait of this technology, and concentration of the carbon allotropes can be used to this end.

Often graphene and other carbon allotropes may be comprised of sheets. The number of sheets may also impact the echogenicity of the allotrope as an ultrasound contrast agent. In one embodiment, the allotrope is one layer thick. In one embodiment, the allotrope is 2-10 layers or sheets. The number of layers and thickness of the sheets correlates with the echogenicity of the composite.

Carbon allotropes can have heterogeneity within the compositions, such as stacked sheets, aggregated nanotubes, and aggregated fullerenes, for example. The nature of the heterogenicity may impact the echogenicity.

Carbon allotropes have well-defined architectures. The method and approach to incorporation of these well-defined architectures within the implant may have an impact on the echogenicity. These methods/approaches can include mixing into the materials via vortexing or sonication for example. Furthermore, the nanometer, micrometer, and millimeter scaled carbon allotropes can be covalently or non-covalently or a combination of covalently and non-covalently bound to the implant, which may alter the echogenicity.

In one embodiment, the allotrope is 0.1 nm to 10 μm in diameter, such as within the range of from 1 nm to 10 μm in diameter, or from 0.5 nm to 5 μm, or from 10 nm to 1 μm, or from 100 nm to 0.1 μm, or from 50 nm to 0.5 μm, or from 1 μm to 5 μm, or from above 0 nm to 10 μm, or from 2 μm to 8 μm in diameter, and so on. In one aspect, the allotrope is preferably 1-10 μm in diameter.

In one embodiment, the soft material is prepared by mixing a carbon-based nanomaterial or allotrope with a polymer-solvent solution. The soft material may also be formed by having two or more polymers cross-link, where the carbon allotrope may be present in one, both, or many constituents prior to their cross-linking.

The carbon-based material or nanomaterial may be added at concentration of 0.1 wt % to 5 wt %, such as from 0.1-0.02 wt %, 0.2-0.3 wt %, 0.3-0.4 wt %, 0.4-0.5 wt %, 0.5-0.6 wt %, 0.6-0.7 wt %, and so on and so forth to 5 wt %, however, it is preferred that the carbon-based material or nanomaterial is added at a concentration of 0.01-0.1 wt %. In embodiments, it is preferred to have the carbon-based material, carbon-based nanomaterial, or carbon-based allotrope present in the end composition in an amount ranging from 10 ng/ml to 100 mg/ml, such as from 50 ng/ml to 50 mg/ml, or from 100 ng/ml to 1 mg/ml, or from 250 ng/ml to 75 mg/ml, or from 500 ng/ml to 30 mg/ml, or from 1 μg/ml to 10 mg/ml, or from 100 μg/ml to 800 μg/ml, or from 400 ng/ml to 500 μg/ml, or from 250 μg/ml to 900 μg/ml, and so forth, including any intermediate range or endpoint.

Once the carbon-based material or allotrope is added, it may be dispersed homogeneously by vortexing, probe sonication, or ultrasonication bath. For example, with respect to ultrasonication, such a procedure may be performed from 10 degrees C. to 70 degrees C., preferably between 20 and 25 degrees C., for 1-10 hours, preferably from 2-3 hours at a frequency set between 1-14 MHz preferably between 5-7 MHz. Ultrasonication is the preferred method for carbon nanomaterial dispersal due to its ability to uniformly disperse the material into the polymer-solvent solution. Uniform dispersion of the carbon-based material is important for preventing agglomeration and keeping cytotoxicity at a minimum.

According to embodiments, the carbon-based material or allotrope is functionalized. Chemical functional groups within materials can dictate hydrophilicity, hydrophobicity, and even amphiphilicity to different conditions. The functional group may directly impact the echogenicity of the material or allotrope as an ultrasound contrast agent. For example, a multi-walled carbon nanotube (MWCN) functionalized with ammonia (NH3) is more echogenic than pristine MWCN.

In embodiments, the carbon-based material or allotrope may be functionalized with one or more of the following: carboxylic acid (COOH) or carboxylic group, amine (NH2), ammonia (NH3) or ammonium, pristine, argon (Ar), silicon (Si), a fluorocarbon, nitrogen (N2), fluorine (F), oxygen, alkyl, cycloalkyl, aryl, alkylaryl, amide, ester, ether, sulfonamide, carboxylate, sulfonate, phosphonate, fluorocarbons, carbonates, nitro, halogens (bromine, chlorine, fluorine), boron, boronic acids, biomacromolecules including sugars and proteins, polymers such as polyethylene glycol (PEG) or pi conjugated polymers, and supramolecular/coordination complexes including metal coordination complexes, and supramolecular complexes (e.g. π-π interactions with aromatics and pi-conjugated materials), or combinations of any of these. These supramolecular complexes include non-covalent interactions such as host-guest chemistry/binding, hydrogen-bonding, van der Waals, and pi-pi stacking with small molecules, oligomers, polymers, polysaccharides, sugars, oligosaccharides, proteins, peptides, oligonucleotides, biomolecules, RNA/DNA, aptamers, biomolecules, derivatives of biomolecules, and other derivatives.

In one embodiment, the composition includes binding partners and/or ligand partners which facilitate cross-linking of the carbon-based material or nanomaterial with the polymer. In embodiments, the binding partners and/or ligand partners which facilitate cross-linking are aromatic compounds such as any substituted or unsubstituted C4 to C10 aromatic compound, optionally with one or more carbon replaced by oxygen, nitrogen or sulfur, including for example naphthalene diimide terminated polymer X linker (telechelic or star polymer).

One reason that carbon materials and allotropes have echogenic properties is due to their mechanical properties, such as their density and velocity of sound, being different than that of biological tissues. The signal for carbon materials and allotropes (their high acoustic impedance) is significantly higher than what is produced by biological tissues (which have low acoustic impedance). This is due to the carbon material and allotropes chemical and physical properties.

By embedding soft materials with carbon materials or allotropes, the soft material may have a broad range of mechanical properties. Different tissues are characterized by their mechanical properties and it is important for implanted materials to be able to interface within the tissue spaces in which they are implanted. By controlling the stiffness of these materials, they are able to mimic native tissues. Additionally, by accessing stiffnesses outside the range of biological tissues, these materials can result in new therapeutics. In addition or alternatively to stiffness, the carbon allotrope may have a direct influence on the soft material's viscosity, thermal conductivity, electrical conductivity, porosity and occlusive properties, and elasticity, or combinations of any of these.

By embedding soft materials with carbon materials or carbon allotropes it may be possible to change the biological properties. These changes can be global and/or occur at the interface of the material and tissue. These biological properties can include induction of different pathways such as angiogenesis, vasculogenesis, neurogenesis, adipogenesis, cellular migration, cellular diffusion, tissue regeneration, tissue growth, irritation, and/or degrees of inflammation.

By embedding soft materials with chemically functionalized carbon materials or carbon allotropes it may be possible to change the environment within and/or around the implant which in turn may impact the echogenicity. The changes for example can be change in local impedance, ion concentration, metal content, proton transport, cation transport, and/or anion transport. The carbon materials can be functionalized or have bound to them any one or more of the following: acids, bases, amino acids, peptides, proteins, antibodies, DNA base pairs, RNA base pairs, oligonucleotides, metal chelators, metal binders, hydrophilic molecules, amphiphilic molecules, lipids, fatty acids, hydrophobic molecules, aromatic molecules (such as benzene, naphthalene, anthracene), conductive polymers, and conjugated polymers. By embedding the soft material with carbon materials or allotropes, the soft material may be imaged, analyzed, and/or tracked by ultrasound. For example, the pore size can be assessed and the implant may be monitored with ultrasound to determine if the implant remains effective at occluding. Furthermore, the ability of molecules and biomolecules to diffuse within and/or from an implanted material's pores is essential for tissue engineering and drug delivery.

Ultrasound may be further utilized to examine, assess, and/or quantify one or more of the mechanical properties of the soft material. In one example, ultrasound is used to determine if the implant remains mechanically durable and/or intact within a bodily tissue, vessel or duct.

In one embodiment, embedding carbon materials or allotropes into the soft material allows for the soft material to have echogenicity over the lifetime of the implant. In one aspect, the lifespan of the soft material, and therefore its echogenicity, may be hours, days, weeks, months, and/or years. Preferably, this invention is particularly useful for soft materials that last greater than 1 year in vivo. Current ultrasound contrast-agents do not have the capability of being ultrasound imageable for greater than 1 year. Usually, ultrasound contrast-agents are used for illuminating bodily tissues or implants during the implantation itself, thus only for seconds to minutes.

Ultrasound may be used to monitor the soft material containing carbon material or allotropes over time and assess the soft material's morphometric properties. This includes length, width, and density of the soft material. This is especially important if the soft material is biodegradable and requires tracking over a period of time. The soft material may begin to degrade at a period of time (for example, at 6 months, 1 year, 1.5 years, or 2 years), and as such, the patient would need confirmation of the soft material's presence within the body. When the implant degrades, the carbon material or allotrope would no longer be embedded and the degrading soft material may lose its echogenicity. This may be useful in signaling when the implant is fully degraded.

In one embodiment, the soft material's composition i.e. polymer(s) may be tuned to impact the life-span of the soft material. In one aspect, the carbon material or allotrope embedded into the soft material itself gives the soft material a longer life-span due to enhanced mechanical properties. In one aspect, the carbon material or allotrope may be tuned to provide a variety of life-spans for the soft material.

### EXAMPLES

1. An algorithm was designed to automatically detect soft materials (specifically, hydrogels) in ultrasound images. The gels comprised various ultrasound contrast agents. The objective was to analyze the echogenicity and homogeneity of various ultrasound contrast agents. Gels were prepared consisting of the following UCA's: argon microbubbles (n=6), chitosan (n=5), and glass microspheres (n=4). The algorithm was run on the images and showed an accuracy of 96.47% (p-value <0.0001). After converting the images to binary images and performing morphological techniques such as smoothing, the Sobel edge detection method was used to find the location parameters of each gel. The extracted ROI was then used to determine the echogenicity and homogeneity. The results showed that glass microspheres were more echogenic than chitosan, which was more echogenic than argon microbubbles. Chitosan had the greatest homogeneity of the three UCA's, but did have aggregation in certain areas of the gel. The argon microbubbles had the least homogeneity. As a result, it was determined that there was a great need for more echogenic, long-lasting ultrasound contrast agents that could be suspended homogeneously within a soft material. As such, carbon-based nanomaterials and allotropes were tested.

2. An implantable soft material was prepared comprising styrene maleic anhydride (SMA) in dimethyl sulfoxide (DMSO). The solution was injected into water to precipitate the SMA implant, followed by suspending the implant in a phantom model. When ultrasound imaged, the implant was minimally echogenic. The same formulation was prepared, except graphene nanoplatelets were added to the SMA-DMSO solution and vortexed. The implant was formed and suspended in a phantom model. The implant was significantly more echogenic under ultrasound. The algorithm described herein was applied on the ultrasound images of the graphene-containing implant. The algorithm detected the implant, isolated the ROI, calculated its width, length, homogeneity, and echogenicity. The echogenicity of this implant was of similar values to previously seen SMA implants containing microbubbles made of polymer or glass. Other formulations can be prepared, varying the functionalization group on the graphene (e.g. NH2, NH3, and/or COOH), and then forming the compositions/implants and imaging them. Finally, the type of carbon nanomaterial or allotrope can be varied in the formulation. These variations include single-walled carbon nanotubes, multi-walled carbon nanotubes, buckyballs, graphene oxide, graphene nanoribbons, and/or fullerenes. All formulations can be imaged in phantom models and analyzed using previously described algorithms to determine the difference in their echogenicity and homogeneity.

3. An implantable soft material was prepared by crosslinking two polyethylene glycol tetramers. The hydrogel was placed in a phantom model and ultrasound imaged. On its own, the implant was minimally echogenic. Next, the same formulation was prepared, except graphene nanoplatelets were added to one of the PEG solutions and the solution was mixed. The implant was formed and suspended in a phantom model. The implant was significantly more echogenic under ultrasound. The algorithm described herein was applied on the ultrasound images of the graphene-containing implant. The algorithm detected the implant, isolated the ROI, calculated its width, length, homogeneity, and echogenicity. The echogenicity of this implant was of similar values to previously seen PEG gels containing microbubbles made of polymer or glass shells. The same procedure was repeated, except the graphene component was added to both starting PEG solutions. The formed implant had significantly higher echogenicity than the implant where only one solution contained graphene. Furthermore, the homogeneity of the implant was better when both components contained the carbon nanomaterial. Other formulations can be prepared, varying the functionalization group of the graphene (e.g. NH2, NH3, and/or COOH), and then forming the compositions/implants and imaging them. Still further, the type of carbon nanomaterial or allotrope can be varied. These variations include single-walled carbon nanotubes, multi-walled carbon nanotubes, buckyballs, graphene oxide, graphene nanoribbons, and/or fullerenes. All formulations can be imaged in phantom models followed by analysis using previously described algorithms to determine the difference in their echogenicity and homogeneity.

4. Accelerated aging experiments can be conducted to measure the echogenicity of soft materials containing long-lasting ultrasound contrast agents over time. Gels can be prepared with a variety of carbon nanomaterials and/or allotropes including graphene powder, graphene nanoplatelets, single-walled carbon nanotubes, multi-walled carbon nanotubes, buckyballs, graphene oxide, graphene nanoribbons, and/or fullerenes. The gels can be placed in buffer to allow for swelling at 37 degrees C. Next, the gels can be placed in vials at varying temperatures (e.g. 25, 37, 45, 55, 70 degrees C.). Ultrasound images can be taken of the gels initially after formation, and once per month at the different temperature groups. With confirmation using the algorithms described herein, it can be shown that the echogenicity of the gels is expected to last over 1-year and eventually, as long as the gels themselves before the gels fully degrade into solution. In addition, the algorithms can be used to characterize the gel's dimensions (e.g. length, width) and their homogeneity. After the gels are completely degraded, it would be expected that the implants could no longer be seen on ultrasound. The systems and methods of the invention and algorithms relating to them can be used to predict and track the speed of degradation of implants, such as implants made using the materials of these examples.

5. Samples of canine vas deferens were placed in a phantom model and ultrasound imaged. The first objective was to determine if the algorithm described herein could detect the inner lumens of ducts or vessels such as the vas deferens. While the algorithm used to detect soft materials generally also worked for the vas deferens, some adjustment was needed. While computationally expensive, active contours were added to the algorithm as well as level set segmentations. As a result, the algorithm was able to successfully identify the inner lumen of the vas deferens. Next, the vas deferens were implanted with soft material containing various ultrasound contrast agents, such as glass microbubbles, chitosan, and carbon nanomaterials. The algorithm was applied on the images of the filled vas. The algorithm successfully isolated the soft material within the vas deferens. This is mainly due to the higher echogenicity of the UCA's in the gel compared to the echogenicity of the smooth muscle layers of the vas deferens. The algorithm could determine the proximal and distal ends of the soft material due to the black space on both sides (empty lumen). This would be clinically relevant for calculating the implant's length and degradation over time.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. An imaging method comprising:
capturing an image or images of an object using an imaging modality;
automatically analyzing the image or images using a computer processor by:
converting the image or images to at least one binary image, and
analyzing the at least one binary image to extract and/or segment one or more regions-of-interest (ROIs) from the at least one binary image.

2. An imaging method comprising:
(a) capturing one or more images using ultrasound;
(b) automatically analyzing the one or more images using a computer processor by:
converting the one or more images to at least one binary image;
identifying from the at least one binary image a plurality of regions-of-interest (ROIs) that may relate to one or more implants, occlusions and/or medical devices;
optionally improving quality of data encompassed by one or more of the ROIs by applying one or more morphological techniques to at least one of the binary images;
evaluating the plurality of ROIs to identify one or more probable ROI candidates according to which of the plurality of ROIs most likely represent the one or more implants, occlusions and/or medical devices;
(c) based on the results of the evaluating, identifying one of the ROIs from the probable ROI candidates as the one or more implants, occlusions, and/or medical devices.

3. The method of claim 2, comprising applying the one or more morphological techniques to at least one of the binary images.

4. The method of claim 2, wherein the ROI is capable of being detected with methods such as the Sobel, Prewitt, Robers, Log, and/or Canny methods.

5. The method of claim 3, wherein one or more of the morphological techniques is chosen from erosion, dilation, filling, template matching, level set segmentation, median filtering, and/or active contours.

6. The method of claim 2, further comprising automatically determining a relative location of, length, width, echogenicity, homogeneity, degradation over time, and/or tissue reactivity of the one or more implants, occlusions and/or medical devices.

7. An echogenic medical implant composition comprising:
a soft, implantable material with one or more carbon-based material, carbon-based nanomaterial, and/or carbon-based allotrope present in an amount sufficient as an ultrasound contrast agent.

8. The composition of claim 7, wherein the carbon-based material, carbon-based nanomaterial, and/or carbon-based allotrope comprises one or more of graphene, graphene powder, graphene oxide, nanoscale graphene oxide, reduced graphene oxide, graphene nanoribbons, graphene nanotubes, graphene sheets, graphene films, granulated graphene, graphene quantum dots, graphene nanoribbons, graphene nanocoils, graphene aerogels, graphene nanoplatelets, carbon nanotubes (single walled, double walled, or multiwalled), nanosheets, nanocones, nanoribbons, buckyballs, and/or fullerenes.

9. The composition of claim 7, wherein one or more of the carbon-based material, carbon-based nanomaterial, and/or carbon-based allotrope has an average diameter in the range of from about 0.1 nm to 10 μm.

10. The composition of claim 9, wherein one or more of the carbon-based material, carbon-based nanomaterial, and/or carbon-based allotrope has an average diameter in the range of from about 1-10 μm.

11. The composition of claim 7, wherein one or more of the carbon-based material, carbon-based nanomaterial, and/or carbon-based allotrope is present in an amount ranging from about 10 ng/ml to 100 mg/ml.

12. The composition of claim 7, wherein one or more of the carbon-based material, carbon-based nanomaterial, and/or carbon-based allotrope is functionalized.

13. The composition of claim 12, wherein one or more of the carbon-based material, carbon-based nanomaterial, and/or carbon-based allotrope is functionalized with one or more functional group capable of providing, dictating, and/or affecting hydrophilicity, hydrophobicity, or amphiphilicity of the composition.

14. The composition of claim 12, wherein one or more of the carbon-based material, carbon-based nanomaterial, and/or carbon-based allotrope is functionalized with one or more functional group capable of providing, dictating, and/or affecting echogenicity of the composition.

15. The composition of claim 12, wherein one or more of the carbon-based material, carbon-based nanomaterial, and/or carbon-based allotrope is functionalized with one or more of carboxylic acid (COOH) or carboxylic group, amine (NH2), ammonia (NH3) or ammonium, pristine, argon (Ar), silicon (Si), a fluorocarbon, nitrogen (N2), fluorine (F), oxygen, alkyl, cycloalkyl, aryl, alkylaryl, amide, ester, ether, sulfonamide, carboxylate, sulfonate, phosphonate, fluorocarbons, carbonates, nitro, halogens (bromine, chlorine, fluorine), boron, boronic acids, biomacromolecules including sugars and proteins, polymers such as polyethylene glycol (PEG) or pi-conjugated polymers, and supramolecular/coordination complexes including metal coordination complexes, and supramolecular complexes.

16. The composition of claim 7, wherein the soft, implantable material comprises one or more of hydrogels, coatings, microparticles, microgels, nanoparticles, nanogels, foams, sponges, electrospun meshes or fibers, microfibers, and/or nanofibers.

17. The composition of claim 16, wherein the soft, implantable material comprises one or more polymers, random copolymers and/or block co-polymers comprising polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers such as polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline (POx), wherein x is any number from 1-5, polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethylene glycol (PEG) or any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or poly(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetrafluoroethylene RFE such as TEFLON® RFE or KRYTOX® RFE, fluorinated polyethylene (FLPE or NALGENE®), methyl palmitate, temperature responsive polymers such as poly(N-isopropylacrylamide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lactide-co-caprolactone (PLCL), and/or chitosan.

18. The composition of claim 7, wherein the soft, implantable material retains a level of echogenicity for days, months, or years, such as for 1-5 years.

19. The method of claim 2, comprising examining, assessing, and/or quantifying one or more mechanical property of the soft, implantable material by way of ultrasound.

20. The method of claim 19, comprising determining by way of ultrasound if the implant remains mechanically durable, efficacious, and/or intact within a bodily tissue, vessel and/or duct.
